(19) United States
(12) Patent Application Publication
(10) Pub. No.: US 2016/0286871 A1
(43) Pub. Date: Oct. 6, 2016

(54) Worker's Knee Pads

(71) Applicant: Muhamed Becirevic, Sparks, NV (US)

(72) Inventor: Muhamed Becirevic, Sparks, NV (US)

(21) Appl. No.: 15/088,614

(22) Filed: Apr. 1, 2016

Related U.S. Application Data
(60) Provisional application No. 62/142,650, filed on Apr. 3, 2015.

(51) Int. Cl. A41D 13/06; A61F 5/01

(52) U.S. Cl. CPC A41D 13/065; A61F 5/0111; A41D 2600/20

(57) ABSTRACT

Knee pads mounted at the lower leg for better comfort and ventilation. Knee pads are mounted to an ankle brace through one or more struts, e.g., without the pressure and discomfort of a mounting strap in inside of knee.

### FIELD OF THE INVENTION

Knee pads to protect the knees of workers doing tasks on the floor of a construction site. The knee pads are mounted at the more comfortable ankle area and positioned over the knee using mounting struts.

### BACKGROUND OF THE INVENTION

There are many styles of knee pads used in athletics and the construction industry. However, the old style knee pads are not typically designed to provide comfort over the course of a work day, e.g., in flooring construction. For example, current knee pads often apply pressure to the back of the knee and are poorly ventilated. Also, the straps of current knee pads are captured as a foreign object on in the inside of the knee as a worker kneels, causing very high pressures on this relatively delicate area.

In Warren, U.S. Pat. No. 7,197,770, Rolling Kneepad Device, a knee pad and shin extension are strapped to the worker at the ankle and knee. Attached to the front of the knee pad is a three wheel dolly with caster wheels. The device is heavy and uncomfortable. The paired straps at the knee pad continuously apply pressure against the nerves, arteries, veins, and tendons at the back of the knee. The discomfort increases at work with the knee bent at an acute angle.

In view of the above, I see a need exists for knee pads that are lighter, do not apply pressure behind the knee, and allow better ventilation. The present invention provides these and other features that will be apparent upon review of the following.

### SUMMARY OF THE INVENTION

The present inventions include, e.g., devices and methods of protecting the knees of workers kneeled on flooring or construction surfaces. The knee pad devices can include an ankle brace with a mounting strap, a knee pad, a first side strut running from the ankle brace to a first side of the knee pad, and a second side strut running to a second side of the knee pad. Alternately, the knee pad can be mounted and positioned using a single strut. In preferred embodiments, there is not a leg mounting device attached to the knee pad, and there is not a leg mounting device attached to a top half of the first strut or the second strut.

In another aspect of the invention, the knee pad device can include an ankle brace comprising a mounting strap adapted to strap around a person's ankle, a knee pad, a first side strut running from the ankle brace to a first side of the knee pad, and a second side strut running to a second side of the knee pad. In this aspect, there is expressly not a strap (or other mounting means interacting at the knee or upper lower leg level) attached to the knee pad. The knee pad can be mounted with the first and/or second strut slidably mounted to the ankle brace.

The first or second strut can be mounted to the ankle brace at a strut mount configured to receive a variable length of the strut, thereby providing for adjustment of the distance between the ankle brace and knee pad. For example, the first or second strut can have two or more perforations and the strut mount have a channel to slidably receive the strut. The channel can have one or more studs adapted to capture one or more of the perforations, so that a desired distance between the ankle strap and knee pad can be selected. In most embodiments, the first strut and/or second strut have a length ranging from 8 inches to 16 inches.

In another aspect of the invention, the knee pad device has an ankle brace comprising mounting strap, a knee pad, and a shin strut running from the ankle brace to the knee pad. In this case, typically there is not a mounting strap at the knee pad and there is not a mounting strap in the top half of the shin strut.

The ankle strap can have an upward extension comprising a strut mount. With the ankle strap laid out open and flat (see FIG. 3) an angle between a center axis of the first strut and a center axis of the second strut ranges from 40 and 80 degrees.

Mounting options exist between the knee pad and ankle brace. For example, the knee pad can be mounted to the first or second strut with a pivotable mount. The struts can be mounted to the knee pad on opposite sides of the knee pad. Optionally the device can have one strut, e.g., a central strut, such as a fixed shin guard. In preferred embodiments, the device does not comprise a shin guard.

The knee pad devices described herein can be worn by a person with the ankle strap tightened around the persons ankle, and the knee pad positioned in front of the person's knee. It is preferred that there is not a strap (or other knee pad mounting means) attached to a top 20% of the device and running around the person's leg.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a surface” includes a combination of two or more surfaces; reference to “strap” includes combinations of straps, and the like.

Unless defined otherwise, all technical and scientific terms used herein have the same meaning as commonly understood by one of ordinary skill in the art to which the invention pertains. Although any methods and materials similar or equivalent to those described herein can be practiced without undue experimentation based on the present disclosure, preferred materials and methods are described herein. In describing and claiming the present invention, the following terminology will be used in accordance with the definitions set out below

An ankle brace as used herein is a brace configured to fit about the lower portion of a lower leg. This includes the ankle itself and the narrowest part of the lower leg just above the ankle. For example the ankle brace is configured to encircle and provide a secure mount at a location ranging from the ankle to the region around the bottom 30% of the tibia. An ankle brace typically grasps the ankle and/or bottom of the lower leg and provides structures onto or into which knee pad supporting struts are mounted. The ankle brace can have significant contact with the top of the foot, e.g., to provide a stable foundation against the weight of the ankle brace system.

A mounting strap is a feature of a brace that is configured to wrap around the ankle or lower leg to mount the knee pad device onto a user of the device. The mounting brace can also make functional support contacts with the top of the foot.

A knee pad is as known in the art. For example, a knee pad is a feature adapted to covers the front of a knee. Typically, the knee pads have a concaved back side contoured to comfortably receive a knee. Usually the knee pads include a tough outer surface to protect the knee from abrasion and other stresses. A knee pad can include extensions (e.g., a shin guard or strut) extending down below the knee, but for purposes of the invention description the “knee pad” refers to only that part configured to cover and protect the front of the wearer's knee area.

A side strut is as described and depicted herein. Struts are typically slender features configured to resist compression and lateral forces so that they can support a knee pad from a brace mount foundation. Side struts of the device are those adapted to run along the inside and/or outside of a user's leg.

A shin guard is as known in the art. Typically a shin guard is a tough layer of material configured to cover the front of a user's leg at the shin area, e.g., protecting against impact, or other injuries. A shin guard is not merely fabric from clothing, e.g., pants.

As used herein, directional terms, such as “upper”, “inner”, “outer”, “lower”, “top”, and “bottom” are as in common usage, e.g., from the orientation of a device on a body standing on earth. Height, width, and depth dimensions are according to common usage.

As used herein, unitary means two features are aspects of the same unbroken piece of material. For example, two features molded together or in the same billet are unitary. Gluing or fixing two separate features together does not make them unitary.

The term “about”, as used herein, indicates the value of a given quantity can include quantities ranging within 10% of the stated value, or optionally within 5% of the value, or in some embodiments within 1% of the value.

### BRIEF DESCRIPTION OF THE DRAWINGS

FIG. 1 is a schematic diagram side view of an exemplary knee pad device as worn by a worker with a bent knee.

FIG. 2 is a schematic diagram front view of an exemplary knee pad device as worn by a worker.

FIG. 3 is a schematic diagram of an exemplary ankle brace laid out flat.

FIG. 4 is a schematic diagram of an exemplary knee pad device strut.

### DETAILED DESCRIPTION

The knee pad devices generally include a knee pad mounted to an ankle brace mount through one or more struts. In use, the ankle brace is mounted to the ankle or lower leg of a worker, and the struts are adjusted for proper length position the knee pad over the worker's knee. There is typically no need for a strap to hold the knee pad in place in front of the knee because the struts orient the pad, and the pad is held against the knee in use by the worker's weight as he kneels practicing his craft.

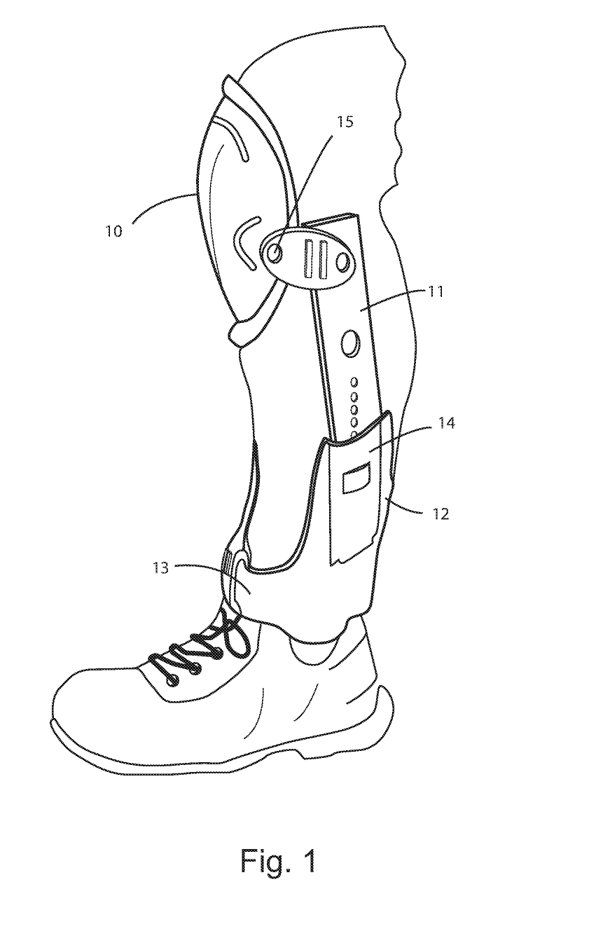

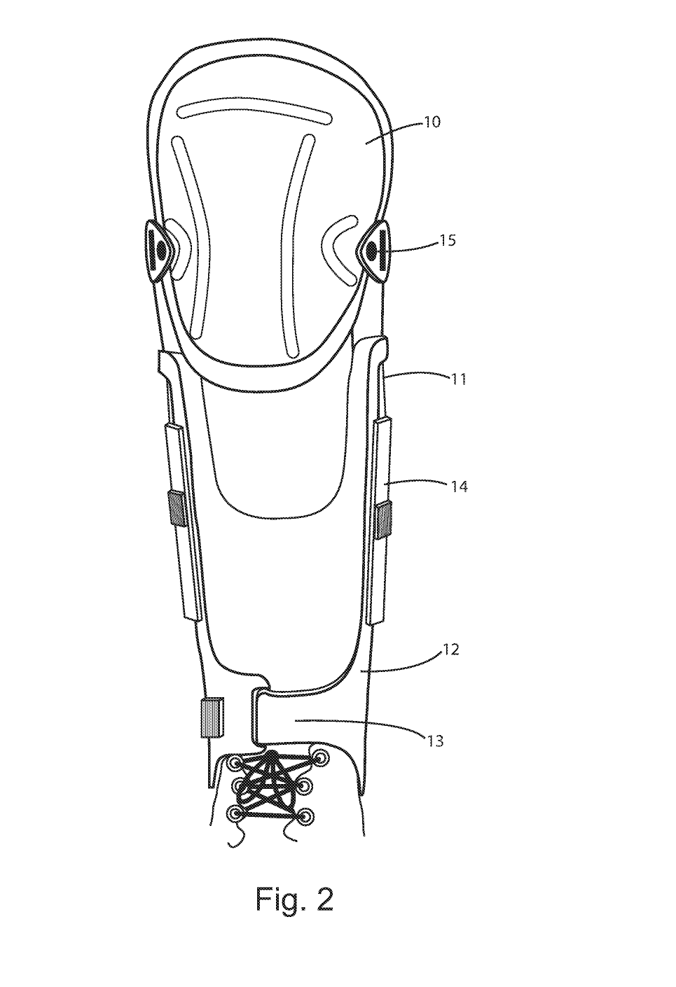

As shown in FIGS. 1 and 2, the knee pad 10 is positioned in front of a worker's knee by struts 11. The struts are slidably mounted in ankle brace 12, which is securely grasping the worker's ankle using mounting strap 13. Positioning of the knee pad can be adjusted by providing struts of different length, or by allowing a slidable strut mount 14, e.g., in the knee pad and/or brace. The knee pad can be pivotably mounted to the struts, e.g., with pivotable mount 15, allowing the knee pad to change relative orientation to the strut, e.g., depending on the angle of the worker's knee.

Knee Pads. The knee pads of the devices provide comfort and protection for the worker's knee as he kneels on a floor surface. The knee pad typically is cup shaped with a padded back side shaped to accommodate the shape of a worker's knee. The front side is typically made of a tough material to take the abuse of sliding on rough surfaces. The knee pad has a mounting component to functionally interact with a complementary strut mount. The knee pad preferably does not include a connection with any kind of feature that wraps around the worker's leg at the level of the knee pad.

The knee pads are dimensioned to comfortably fit over the front of a knee, particularly with the topography of a flexed knee, as presented while a worker is kneeling. The knee pads typically have a length and/or width ranging from 6 cm or less to 20 cm or more, from 7 cm to 17 cm, from 8 cm to 15 cm, or about 12 cm.

The front of the knee pad is usually made from a hard or tough material. Typically the knee pad front is fabricated from plastic, leather, metal, and/or a rubber. Vent holes may be present to provide additional ventilation.

The inside of the knee pad is usually provided with a tough comfortable material, such as, e.g., a fabric, foam, styrofoam, matting, sheepskin, silicone rubber, and/or the like. Typically, the padding material is laminated onto the back of the tough front, e.g., with an adhesive or stitching.

The knee pad has a means to be mounted to the upper end of one or more struts. Usually, the mount means allows for some pivoting of the pad about a horizontal axis. In one aspect, the knee pad can include a stud on a side (or on each side) of the tough pad front. The top of the strut (or in the upper half of the strut), or an intermediate mounting connector, can have a complementary hole or slot that pivotably receives the stud. Alternately, the top of the strut can have the stud and the connector or pad have the hole or slot. Optionally, the top end of the strut is fixed to the knee pad, or unitary with the knee pad. The mount between knee pad and strut can be releasable or permanent.

Knee pads can have lower extensions that protect the worker's shin. In one embodiment, the a shin extension can be pivotably mounted to articulate from the bottom of the knee pad cup. The shin extension can extend down from the knee pad cup10%, 25%, 50% 75% or 100% of the way to the ankle. In some embodiments, the lower end of the shin extension can be connected to the ankle brace. The length of the shin extension can be adjustable, e.g., by having alternate connectors along the length of the shin extension to interact with a complementary knee pad mount. The shin extension can be made of similar materials to the knee pad, with a light material, such as plastic foam being preferable.

Ankle Braces. The ankle brace provides a means of grasping the worker, and to provide a mount for one or more struts. The ankle brace wraps around the worker's ankle area, and/or the lower quarter of the worker's lower leg, to provide a secure mount to position the strut(s) in an orientation presenting the knee pad in front of the worker's knee. Although the term ankle brace is used herein, the brace can be configured to mount anywhere from the top of the foot, to the ankle, to the bottom of the lower leg. Typically the brace has a strap to grasp at the top of the ankle, and upward extensions on the sides to mount the struts and spread the mount pressures.

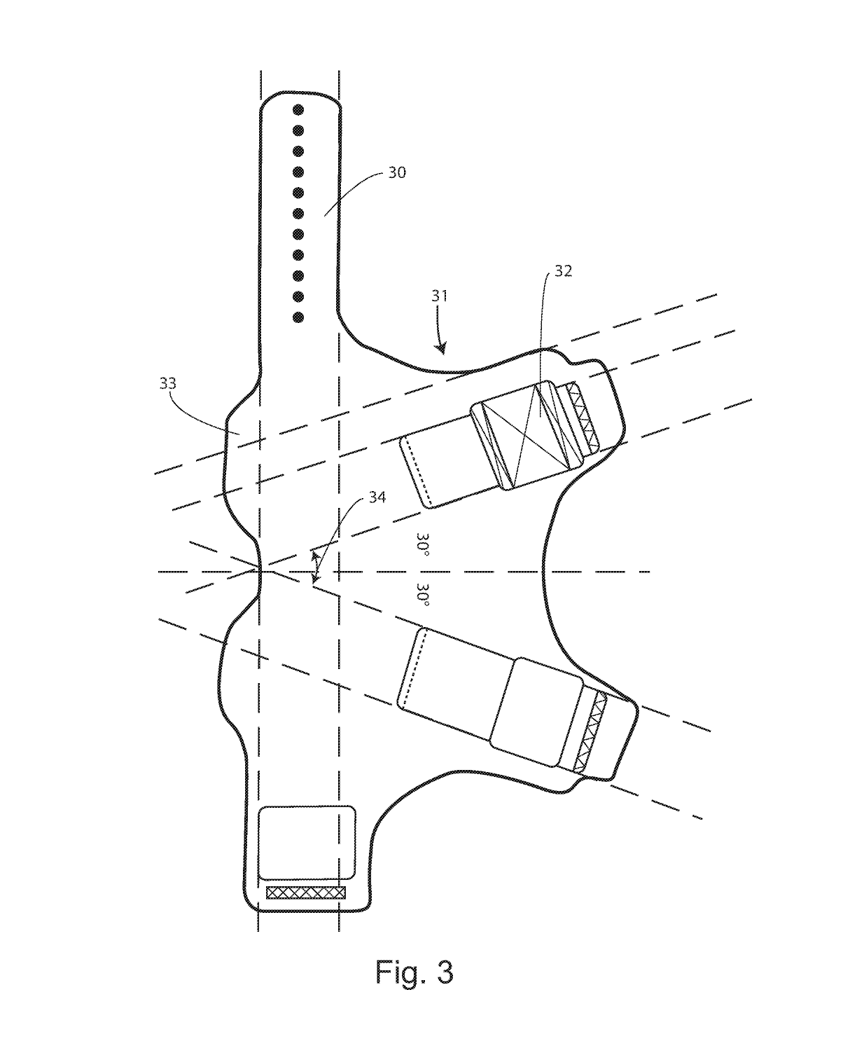

In one aspect of the invention, the brace has a leg mounting strap 30 and an upper section 31 with strut mounting fixtures 32, as shown in FIG. 3. The brace can include lower lobes 33 to provide additional lateral support, in use.

The ankle brace wraps around the worker's lower leg to support the rest of the device in functional relation to the worker's leg and knee. In some aspects, the brace may not wrap all around the worker's lower leg, but in most embodiments the ankle brace is configured to surround the worker's leg (an ankle being part of a leg). For example, the brace can include a strap that can go around the worker's leg and bind the brace to the leg. The strap can be a long flat flexible feature with a means to encircle the leg. For example, the strap can be a belt with a buckle, a strap with tie laces, a strap with snaps, a strap with hook and loop connectors, an elastic strap, a perforated belt cooperating with a buckle having studs fitting the perforations, a clip and lever device (e.g., as found on roller blades and ski boots), and/or the like. The strap feature of the brace can have a length adequate to surround the worker's lower leg, e.g., from less than 15 cm to more than 50 cm, from 20 cm to 40 cm, or about 30 cm. In preferred embodiments, the end of the strap is tucked into the brace, e.g., through a slot, so that it is not exposed to be snagged by workplace objects.

In certain embodiments, the struts mount to the brace at one or more strut mounts. In order to allow adjustment for the distance between the brace and knee pad, it is useful to have a strut mount that allows capture at more than one position along the length of the strut. For example, as shown in FIGS. 1 to 3, the brace can have a channel into which the strut can slide. The channel can include capture means, such as, e.g., a clamp, lace and holes, snaps, studs matching stud holes, and/or the like. Depending, e.g., on the size of the worker wearing the device, the struts can be slid more or less into the strut mounting channels and fixed at a desired length. Alternately, the strut mounts can be simply locations on the brace where struts are fixed, or the struts can be unitary to the brace (e.g., with length adjustment elsewhere along the strut).

In embodiments wherein there are at least two struts they can be mounted to, e.g., the back, back/side, or sides of the brace. Often the struts diverge up from each other. For example, as shown in FIG. 3, the strut mounts direct the struts at an angle away from each other, when viewed with the brace laid out flat in a plane. The angle 34 (measured according to the central axes of the struts or axis of the mount channel) at which the struts diverge can range from less than 10° to more than 90°, 30° to 75°, 45° to 70°, or about 60°. Alternately, the struts can be parallel to each other, or converge slightly upwardly.

Struts. One or more struts can be based at an ankle brace strut mount and extend upwardly to support and position the knee pad. Preferred struts are light and strong. In some embodiments, there is one strut running from the ankle brace to the knee pad. Usually, there are two or more struts, e.g., to provide more lateral support, without interfering with the worker's shin area.

A single strut can be an axial cut cylindrical section arranged to curve around the front of the worker's shin. The strut could be perforated to lighten and increase ventilation.

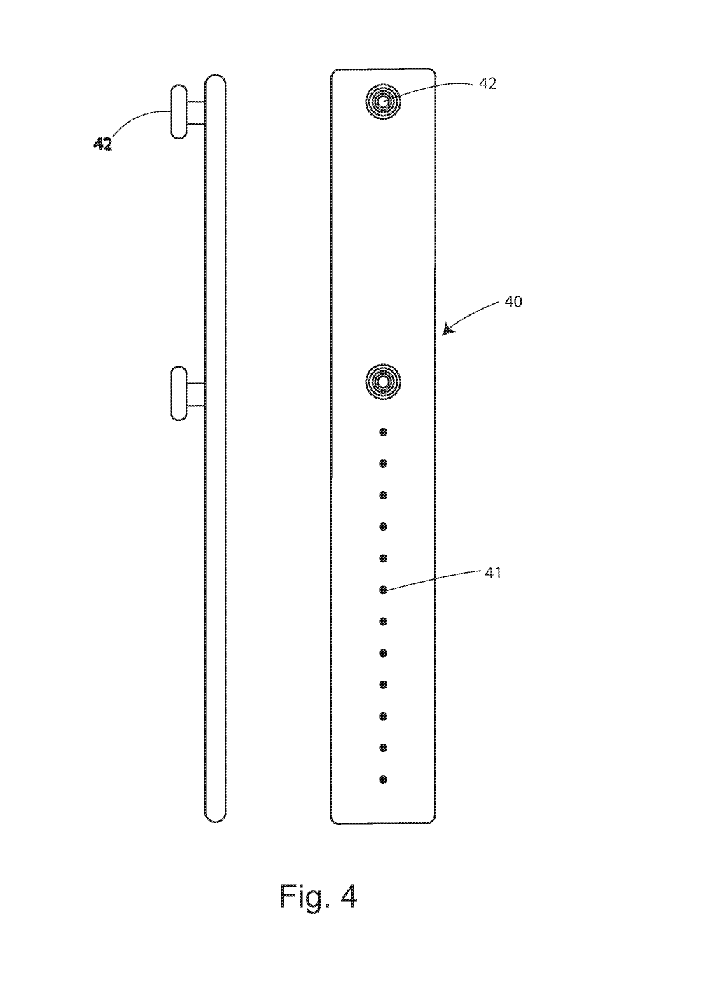

Usually, the devices include two or more struts, arranged so that one strut runs up the inside of the lower lag and another up the outside of the lower leg (e.g., as shown in FIGS. 1 and 2). A typical strut is shown in FIG. 4. The strut 40 includes perforations 41 that interact with capture studs in a strut mount of an ankle brace. The strut includes one or more knee pad mounting pegs 42 that can provide a pivoting mount to the knee pad, or to a mounting connector.

The struts range in lengths appropriate to offset the knee pad the desired distance from the ankle brace, positioning it in front of a particular worker's knee. Often the strut is provided long enough for the tallest expected worker and a slidable mount is used to adjust down for shorter workers. Typically struts range in length from less than about 15 cm to more than 70 cm, from 30 cm to 55 cm, or about 40 cm.

The struts can be fabricated from light and strong materials. It is preferred the materials be rugged with some substantial elasticity or flexibility. The struts can be made from, e.g., plastics, composites, metals, fiber glass, hard rubber, natural materials, and/or the like.

### EXAMPLES

The following examples are offered to illustrate, but not to limit the claimed invention.

### Example 1

### Using the Knee Pad Devices

In use, the device was mostly preassembled, e.g., with the bottom of the struts in the brace strut mounts and the tops of the struts pivotably mounted to the knee pads. The brace strap (or other mounting means) was open to receive the leg bottom at or near the ankle. The worker closed the ankle brace about his ankle or lower leg (e.g., around a boot top) and tightened it to provide a firm foundation to support the device. If the knee pad did not properly align with the worker's knee, the strut mounts could have been released to allow the struts to slide in the mount channel to a position locating the knee pad in front of the worker's knee. When the knee pad was properly aligned, the strut mounts could be locked to retain the desired distance between the brace support and the knee pad.

There is no strap, or even other knee pad mounting means, running from the knee pad or top 30% of the device (measured from bottom of brace to top of knee pad).

When standing, the knee pad was spaced somewhat from the worker's knee. The knee experienced no pressure from the device, and air flowed freely about the area. There was some pressure around the ankle area, but the worker experienced no stress because the bone and tendon in this area are less subject to pressure stress, and the boot top and brace further distribute the constrictive forces.

The worker could kneel and do flooring work with the comfort of the knee padding and protection of the knee pad outer shell. The device is light and comfortable, even over extended work periods. The device was simply removed by unbuckling the ankle brace.

### Example 2

### Alternate Devices

Although the present devices are described largely as a combination of pad/strut/brace, inventive sub-combinations have substantial utility.

For example, the combination of the brace and one or more struts can be used to mount and/or position elements other than knee pads. The alternate elements can be positioned in front of the knee and/or at other locations along and/or around the leg.

In another alternate, the pad/strut pair can have utility without interacting strictly with an ankle brace. For example, it is envisioned that an alternate foundation can receive the struts to locate the pad. A pant leg, boot upper, or boot top can have mounting features (such as pockets or channels) to functionally receive the bottom end(s) of one or more struts.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A knee pad device comprising:
an ankle brace comprising mounting strap,
a knee pad;
a first side strut running from the ankle brace to a first side of the knee pad; and,
a second side strut running to a second side of the knee pad.

2. The device of claim 1, wherein there is not a leg mounting feature attached to the knee pad.

3. The device of claim 2, wherein there is not a leg mounting feature attached to a top half of the first strut or to the second strut.

4. The device of claim 1, wherein the first or second strut is mounted to the ankle brace at a strut mount configured to receive a variable length of the strut, thereby providing for adjustment of a distance between the ankle brace and knee pad.

5. The device of claim 4, wherein the first or second strut comprising two or more perforations and the strut mount comprises a channel to slidably receive the strut, the channel comprising one or more studs adapted to capture one or more of the perforations, thereby selecting a distance between the ankle strap and knee pad.

6. The device of claim 1, wherein the ankle strap comprises an upward extension comprising a strut mount.

7. The device of claim 1, wherein, with the ankle strap laid out open and flat, an angle between a center axis of the first strut and a center axis of the second strut is in the range from 40 to 80 degrees.

8. The device of claim 1, wherein the knee pad is mounted to the first or second strut with a pivotable mount.

9. The device of claim 1, wherein the first side of the knee pad is opposite the second side of the knee pad.

10. The device of claim 1, wherein the device does not comprise a shin guard.

11. The device of claim 1, further comprising a shin guard extension extending down from the knee pad.

12. The device of claim 1, worn by a person with the ankle strap tightened around the persons ankle, and the knee pad positioned in front of the person's knee; wherein there is not a strap attached to a top 20% of the device.

13. The device of claim 1, wherein the first strut or second strut comprises a length ranging from 8 inches to 16 inches.

14. A knee pad device comprising:
an ankle brace comprising mounting strap adapted to strap around a person's ankle,
a knee pad;
a first side strut running from the ankle brace to a first side of the knee pad; and,
a second side strut running to a second side of the knee pad;
wherein there is not a strap directly attached to the knee pad.

15. The device of claim 13, wherein the first or second strut is slidably mounted to the ankle brace.

16. A knee pad device comprising:
an ankle brace comprising mounting strap;
a knee pad; and,
a shin strut running from the ankle brace to the knee pad;
wherein there is not a mounting strap at the knee pad and there is not a mounting strap in a top half of the shin strut.
